(19) United States
(12) Patent Application Publication
(10) Pub. No.: US 2009/0192500 A1
(43) Pub. Date: Jul. 30, 2009

(54) MEDICAL DEVICE FOR ACTIVE DRUG DELIVERY VIA SOLAR ENERGY

(76) Inventor: Eva Cortez, San Francisco, CA (US)

Correspondence Address: John Bargero, P.O BOX 471857, SAN FRANCISCO, CA, 94123, US

(21) Appl. No.: 12/360,855

(22) Filed: Jan. 28, 2009

Related U.S. Application Data
(60) Provisional application No. 61/062,487, filed on Jan. 28, 2008.

(51) Int. Cl. A61M 37/00

(52) U.S. Cl. 604/890.1; 604/304

(57) ABSTRACT

A surface wearable or implanted medical device suitable for delivering therapeutic agents to the body as well as monitoring and analyzing analytes in bodily fluids. The device utilizes a photovoltaic cell or miniature solar panel as an energy source in conjunction with microtubules or membranes, a therapeutic agent or analyte reservoir, an adhering structure, and a means of regulating and monitoring the agents and analytes. The device is adhered to the tissue of the recipient and by controlling the polarity of the current between the tissue and reservoir the flow of agents into the body can be controlled and regulated, just as the rate of removal of analytes from the body can also be regulated. This regulating, analyzing, and monitoring can be controlled by the absence or presence of light or may involve a computerized system that utilizes a transmitter and receiver or visual display system.

### PRIORITY FILING

This application is claiming the filing date of Jan. 28, 2008 of provisional patent application Ser. No. 61/062487.

### FIELD OF INVENTION

This invention relates to a patch-like device to be used for drug delivery using solar energy to create a polarized electrical potential gradient to migrate therapeutic agents into and to remove or detect analyte in the subject's body.

### BACKGROUND OF THE INVENTION AND RELATED ART

The administration of therapeutic agents, such as drugs, to patients, whether human or animal, requires the introduction of compounds into the blood stream of the host. Traditional methods of sub-dermal application require the use of a needle or syringe to penetrate the subject's skin and inject the agent, such as a traditional vaccination. Conversely, in the event of monitoring one's blood, such as in the case of glucose monitoring of a diabetic, a small pinprick is requires to retrieve a sample.

Another method of supplying or removing elements from the host's blood stream utilizes microtubules. Microtubules are very small needle-like probes that penetrate into the body through the skin. Microtubules range in diameter roughly from 10 nm to about 200 nm and when applied to the skins are virtually painless. Typical microtubules are of the type marketed by Pelikan (Palo Alto, Calif.) and/or Kumetrix (Union City, Calif.) see also U.S. Pat. No. 6,503,231. Agents or analytes are either introduced or removed from the blood stream through a combination of a reservoir and fluid pressure gradient between the host and the reservoir, such as a micro-pump. Although this method overcomes some of the drawbacks of tradition needles, it still has shortcomings in the arena of regulating the dosages.

A further method of introducing agents into the blood stream is the use of a trans-dermal patch, such as that used in the cessation of smoking utilizing nicotine. This method also has limits, such as the size of the agent, for example, insulin will not pass through the skin. Also, this patch may be susceptible to variations in heat that will affect the absorption rate and trans-dermal transfer.

Traditional injections generate millions of disposable needles, which enter into the waste stream, and are detrimental in creating a greener environment because they increase disposable waste. Furthermore, there is great risk in infection from contaminated used needles, not only in the waste stream, but also to the administrator or nurse who may be subjected to unwanted pricks.

These aforementioned solutions do not address personalized medicine such as the rate of absorption and the ability of the individual to metabolize the amount of therapeutic agents. By monitoring the levels of biological agents taken from the blood, these observations can be used to infer how much dosage a patient is able to handle, that is the amount is designed for individual body metabolism, and adjust the dosages accordingly reducing adverse effects and allowing the patient to respond better to treatment.

The current solutions do not address the patient's status in real time. Understanding what the body is doing now, real time, can give information how the body will metabolize the drug, and can be an indicator how the body will react to the drug given. The efficacy changes person to person. Presently it is common procedure to over medicate or over dose to ensure that patient's symptoms are alleviated. Dosage beyond the body's ability to absorb the drug causes a multitude of dysfunctions and side effects that influence the function of other organs including mental states. Metabolites of pathways and biomarkers of precursors ideally can be used as parameter data in a bioinformatics system incorporating pharmacogenomics and pharmacokinetic information to determine levels of toxicity and dosing. Looking for markers in the blood stream in real time helps the pharmaceutical industry address real time therapeutics accurately not as a symptomatic end point determining states. Also, this would allow patients to enter clinical trials in a safer fashion with information of some precursor, or predisposition state of individual foreknown. Ideally this would allow our pharmaceutical medicines to get through FDA testing and to market faster with a minimal cost to the people who need it the most.

The pharmaceutical industry spends millions of dollars to get therapeutic treatments to persons. Efficacy in drug dose and formulation of drug stability costs a lot in R&D and more in clinical trials before a drug can get FDA approval. Current modes of drug delivery use time released formulas of drugs in pills or through timed released patches. These methods do not take into account the patient metabolism, metabolites or genetic predisposition or current medication that other drugs may be present. Removal of the additional agents utilized to govern the rate of release into the bloodstream of the therapeutic drug could reduce the R&D formulation time. These additional addatives may negatively affect the stability and shelf life of the therapeutic agent.

Current solutions do not effectively address point of care issues where millions of doses of antibiotics are needed during flu season or other epidemic outbreaks. According to the public health departments, immunities of strains of virus and bacterial is still of a national concern. The ability to give out self administered therapeutic agents to control spreads of viral and biological agents, has tremendous potential applications for developed nations and especially the underdeveloped nations where medical staff and facilities are not available to control the deaths of simple infections.

Additionally, there may be instances when a patient that self-administers medication such as persons with mental disabilities may forget to take their timely dosage due to lack of understanding, costs, forgetfulness, feebleness, and unable to read, or comprehend instruction, or even to open the medical container.

Furthermore, there are some instances when the administration of the agent or the monitoring of the analyte should be proportional to the amount of light available, to mimic the waking/sleeping patterns of the wearer. For example the administration of certain drugs, like Ritalin require higher dosages to the patient when he is awake as opposed to when he is sleeping. Similarly, certain agents that prevent sun-bun, such as lycopene and beta-carotene would be advantageously delivered to the user in proportion to his exposure to the sun.

Consequently, there is a real need for the delivery of antibiotics where there is no doctor, or medical training required for staff to administer the immunity, allowing millions of people to get immunization in rural areas where there are not medical facilities.

Although these modes and methods have served humanity well in the past, there is an opportunity to improve on the delivery of drugs and monitoring analytes in patients.

It is therefore an object of the present invention to provide a drug delivery and analyte monitoring device that is solar powered, user applicable, relatively painless, relatively small, easily distributed, and has a long storage life.

### SUMMARY OF THE INVENTION

A surface wearable or implanted medical device suitable for delivering therapeutic agents to the body as well as monitoring and analyzing analytes in bodily fluids. The device utilizes a photovoltaic cell or miniature solar panel as an energy source in conjunction with microtubules or electrolytes, a therapeutic agent or analyte reservoir, an adhering structure, and a means of regulating and monitoring the agents and analytes.

The monitoring and the regulation of these agents and analytes are governed by an electrokinetic phenomenon, such as electrophoresis and electro-osmosis for example. These processes require a power supply to create a polarized field between the tissue and the agent or analyte. This potential difference allows the agents and analytes to become motive or to interact with the patient, capable of transferring drugs to the patient's bloodstream through tissue, such as skin or other membrane or if the polarity is reversed, removing substances from the bloodstream.

The device is adhered to the tissue of the recipient and by controlling the polarity of the current flow; the flow of agents into the body can be controlled and regulated, just as the rate of removal of analytes from the body can also be regulated. This regulating, analyzing, and monitoring can be controlled by the absence or presence of light or may involve a computerized system that utilizes a transmitter and receiver or visual display system.

Because the device utilizes a photovoltaic device, under conditions where there is no light the patch does not dispense the agent because there is no voltage being generated this mimics the natural cyclic patterns of the body. While other embodiments contemplate an electrical storage device, such a battery to store the electrical potential when there is no light source available, this may be advantageous in monitoring procedures. An appropriate patch can be designed for the type of therapeutic protocol of delivery and its application to that individual patient.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details; in other instances well known processes and operations have not been described in detail, in order not to unnecessarily obscure the present invention. Accordingly, the drawings and description are to be regarded as illustrative in nature, and not as restrictive.

### DESCRIPTION OF THE DRAWINGS

Taking the following specifications in conjunction with the accompanying drawings will cause the invention to be better understood regarding these and other features and advantages. The specifications reference the annexed drawings wherein:

FIG. 1 is a perspective side view of the device.

FIG. 1a is analogous to FIG.1 with the addition of a monitor and transmitter

FIG. 2 is an exploded side view of the device incorporating microtubules.

FIG. 3 is a perspective side view of a microtubule with internal resin bonds

FIG. 4 is a perspective side view of a microtubule with external resin beads.

FIG. 5 is a perspective side view of a collapsible microtubule structure.

### DESCRIPTION OF PREFERRED EMBODIMENTS

While describing the invention and its embodiments, various terms will be used for the sake of clarity. These terms are intended to not only include the recited embodiments, but also all equivalents that perform substantially the same function, in substantially the same manner to achieve the same result.

The terms “Agent” and “Therapeutic Agent” as used herein refers to compounds that are useful in or appropriate for treating a disease associated with a particular biological anomaly indicative of disease, e.g., disease marker analyte. Therapeutic agents are any therapeutic substance for the treatment of diseases including for example: pharmaceutical compounds that are preferably delivered locally such as chemotherapeutics, steroids, therapeutic nucleic acids including DNA, RNA, double stranded RNA (by means of RNA interface) and antisense RNA, or proteins such as immunoglobulin, growth factors, anti-inflammatory agents, or enzyme inhibitors, etc. It is further conceivable that the “agent” used in a broader term may be adopted to mean any molecule or substance or compound transferred into the tissue, regardless of its therapeutic value.

The term “analyte” as used herein refers to antibodies, serum proteins, cholesterol, polysaccharides, nucleic acids, drugs and drug metabolites, metals, lead contaminates, or toxins or pesticide residue, narcotics, poison elements etc., found in bodily fluids and tissues of the body. In another embodiment, the analyte is any biological analyte, marker, gene, protein, metabolite, or hormone or combination therein indicative of a biological state desirable for analysis to determine a physical state or condition.

“Biologicals” are agents or products that are precursors or products of an undesirable component such as lead metals, mercury toxins, pesticides, HIV, magnetic mesenchymal cell clusters, biomarkers, prions of disease, surface membrane proteins, cholesterol, and differentiation of cell stages.

“Interact with,” as used herein refers to the ionic, covalent or hydrogen bonding, protein binding, nucleic acid hybridization, magnetic or hydrophobic attraction or other detectable and/or quantifiable association of an analyte and a bioactive agent on the electrophoresis. While, “Differentially interact with,” refers to the fact that a disease marker biological analyte will interact with a bioactive agent differently than a biological analyte indicative of normal physiology.

“Microsphere” or “beads” or “particles” or grammatical equivalents herein are meant small discrete particles. The composition of the beads will vary, depending on the class of bioactive agent and the method of synthesis. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphited, titanium dioxide, latex or cross-linked dextrans such as Sepharose, cellulose, nylon, cross-linked micelles and teflon may all be used. “Microsphere Detection Guide” from Bangs Laboratories, Fishers Ind. is a helpful guide, and is incorporated by reference in its entirety. The beads need not be spherical; irregular particles may be used. In addition, the beads may be porous, thus increasing the surface area of the bead available for either bioactive agent attachment or tag attachment. The bead sizes range from nanometers, e.g. 100 nm, to millimeters, e.g., 1 mm, with beads from about 0.2 micron to about 200 microns being preferred, and from about 0.5 to about 5 microns being particularly preferred, although in some embodiments smaller or larger beads may be used. Preferably, each microsphere has a bound affinity agent.

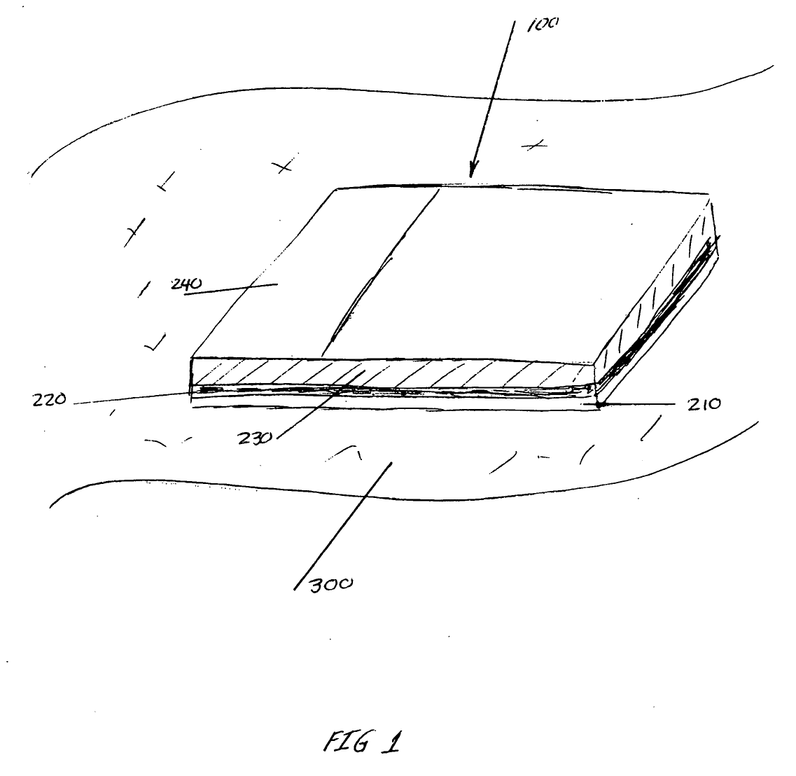

FIG. 1 is a perspective side view of the device generally referenced by the numeral 100 incorporating membrane 210, an insulating barrier 220, a reservoir 230 and a photo-voltaic cell 240. The device 100 is generally arranged as a patch-like structure or very large bandage, such as to cover large wounds. Although, in the preferred embodiment the power source is a photo-voltaic cell 240 it is conceivable and possibly desirable at times to use an ordinary battery or voltaic cell of other construction. The insulating barrier 220 provides electrical insulation as well as is a semi-permeable membrane to allow the passage and transference of agents and analytes to and from the tissue. The membrane 210 has adhesive properties, yet allows the passage of agents and analyte through it, and is placed in contact with the skin or other tissue 300 of the subject, patient, host, etc . . . and allows for the transport of agents into the tissue 300.

This medical device 100 utilizes the reservoir 230 to house or contain therapeutic agents. The photovoltaic cell 240 is electronically connected to the reservoir 230, creating an anode end (+) positively charge component, and to the membrane 210 on the skin surface creating a cathode or (−) negatively charged tissue area. These therapeutic agents are charged and will migrate in the field one location to another in the direction as a characteristic of their charge in the electrical field. The agent's rate of release is related to the rate profile set by the electrical output; just as shifting the electrical polarity will change the direction of this flow.

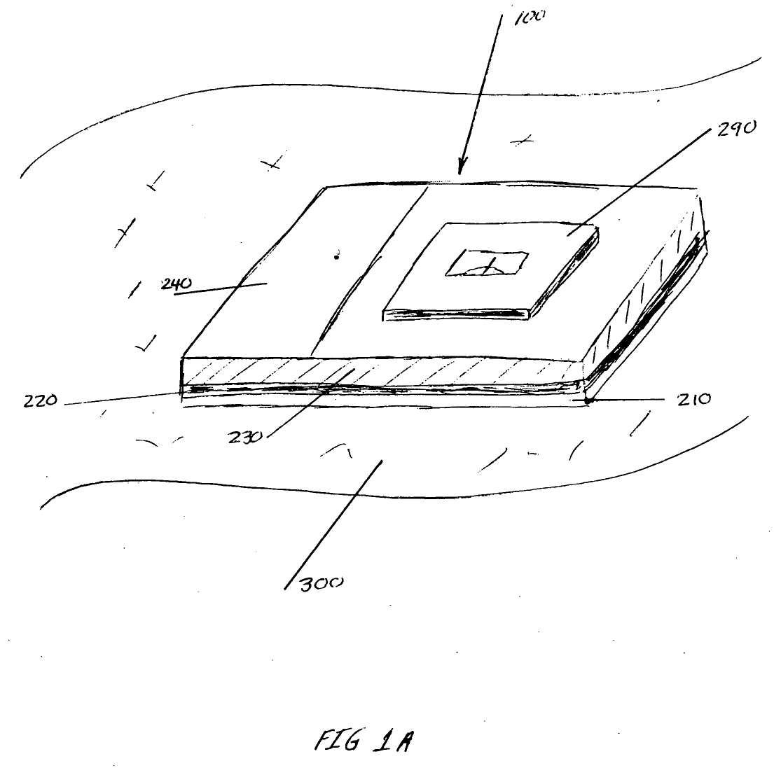

FIG. 2 is a perspective side view of the device generally referenced by the numeral 100 incorporating microtubules 200, an adhesive barrier membrane 215, an insulating barrier 220, a reservoir 230 and a photo-voltaic cell 240.The microtubules 200 supply or remove elements from the host's blood stream to and from the reservoir 230.

This alternative embodiment utilizes microtubules 200 which are needle-like probes that penetrate into the body through the skin or sub-dermal preferable in areas of high capillary density. Microtubules 200 are roughly tubular or cylindrical in shape with a hollowed out internal portion and an external portion or body, about the size of a human hair and have an integrated reservoir. In a preferred embodiment, the microtubules 200 are constructed out of silica and can range from about 10 microns to about 200 microns, preferably about 50 to 150 and most preferably 100 microns in diameter, making their application to the skin virtually painless. The microtubules 200 have two ends, one is attached to the reservoir, while the opposite end or tip penetrates the skin of the host.

In this embodiment the device 100 utilizes the reservoir 230 to house or contain therapeutic agents. The photovoltaic cell 240 is electronically connected to the reservoir 230, creating an anode end (+) positively charge component, and to the microtubule 200 implanted into the tissue or the skin's surface creating a cathode or (−) negatively charged tissue area. These therapeutic agents are charged and will migrate in the field one location to another in the direction as a characteristic of their charge in the electrical field. The agent's rate of release is related to the rate profile set by the electrical output; just as shifting the electrical polarity will change the direction of this flow. Since the microtubules are submerged under the skin surface they interact directly with the blood stream capillaries to collect unwanted biologicals, toxins, metals and carry them out of the body.

In the preferred embodiment there is at least one reservoir 230 that may be used for either storing the agent or combination of various agents to introduce into the tissue, or to store matter taken from the tissue, it is contemplated that a series of reservoirs 230 may be used in the event of segregating agents and matter removed or inserted into the tissue or for other reasons.

In an embodiment the reservoir 230 can be the site of measurements taking advantage of colored pH indicator markers to determine the concentration remaining or of accumulated products. An off the shelf conductivity meter can be used to inference the concentration of ions present, thus translating voltage readings into concentration levels. Several time points can determine a rate profile for the individual which can serve as a biometric analytical factor to help understand the metabolism profile for that particular patient. Monitoring the release of the therapeutic agents can reflect on the amount absorbed by the blood stream, where a therapeutic agent releasing profile can determine absorption rates. In a further embodiment of this aspect of measuring the agents from the body can become an indicator whereas, the analyte is indicative of disease.

Personalized medicine relates to pharmacogenetics and pharmacogenomics dealing with the genetic basis underlying variable drug response in individual patients. Measuring the reservoir 230 of the patch is a real time indicator of what the patients' metabolic state is now taking into account the predisposition of illness and disease.

Measurements taken at the reservoir 230 can qualitatively and/or quantitatively “detect” analytes in the bodily fluids. Preferably, such detection occurs periodically. Most preferably, it occurs in real time. In one embodiment, the analytes are present in micromolar to nanomolar concentrations and are highly potent chemotherapeutics, such as aminoglycocides or antibiotics, e.g., vancomycin, for which minute to minute monitoring is highly desirable because the analytes have narrow therapeutic ranges. This can target the rate of absorption and the ability of the individual to metabolize the amount of therapeutic agents by monitoring levels of biological agents taken from the blood, where by the observations can be inferred how much dosage a patient is able to handle, that is the amount is designed for individual body metabolism, which would reduce side effects and allow the patient to respond better to treatment.

FIG. 1a demonstrates an embodiment of the device 100 wherein a monitoring circuit and transmitter 290 are connected to the reservoir 230 and the photo-voltaic cell 240 in order to measure the voltage of the contents of the reservoir 230 and thereby the status of the patient and then transmit that information to an outside source, such as a computer or similar database in real time.

In an alternative embodiment the reservoir 230 stores therapeutic agent until it is directed by the biorecognition device upon detection of a disease marker, to release therapeutic agent in a controlled fashion, e.g., receives instruction as to release rate and quantity of agent to be released. Alternatively, a single release rate or dose may be programmed into the device. The reservoir can contain a mixture of one or more therapeutic agents. Alternatively, the device can comprise several reservoirs of one or more therapeutic agents. Preferably there are pluralities of reservoirs.

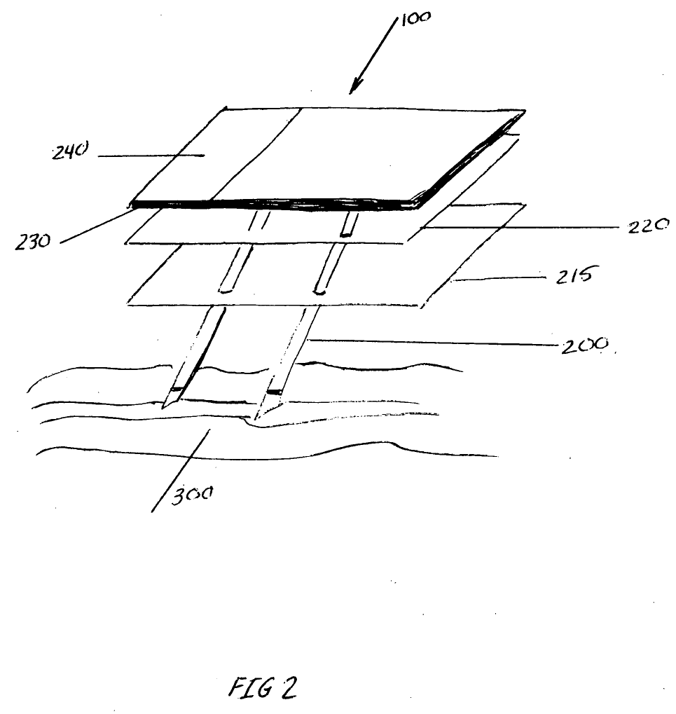

FIG. 3 is a perspective view of a preferred embodiment where at least one of the microtubules 230 are coated on its external surface with sphere-like beads or resins 250 that can have a functional groups or amino acids serving as a binding tag, where as specific tags attached to the bead surface, where the functional groups tags serve to bind desired components from the body or bloodstream.

The microtubule 200 has a charged tip or ring 280, capable of creating a potential where the bound components are electrically migrated via microtubules 200 to the reservoir 230 at the surface of the skin. The charged ring 280 can be either positive or negative and can serve as an anode or cathode which will determine the flow direction of substances into or out of the body of the user. The charged ring 280 can then act as either an anode or a cathode.

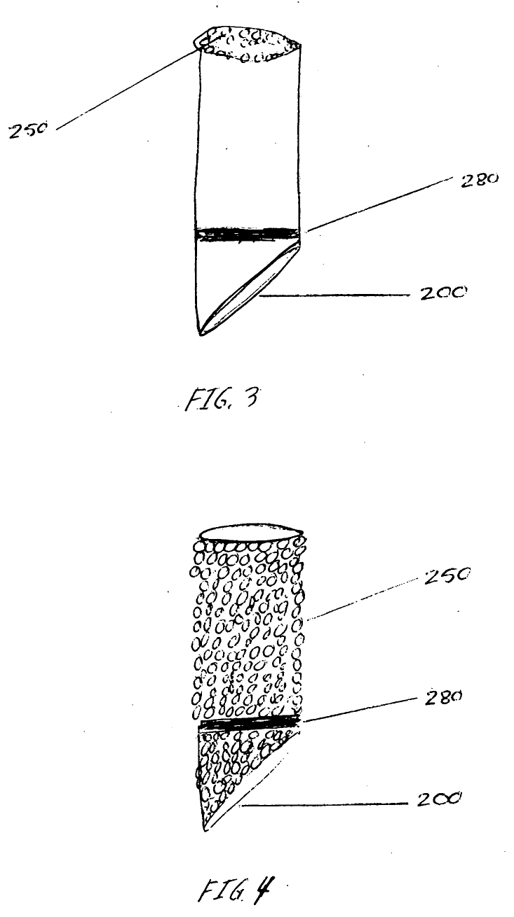

FIG. 4 is a perspective view of a preferred embodiment where at least one of the microtubules 200 has its internal surface coated with sphere-like beads or resins 250, which whether coated or internally packed within the needle-like microtubule 200 acts similar to chromatography concept where as passing bodily fluids interacts with the resin 250 functional groups and the specific “biological” from the body attaches to the resin 250 and remain in the stationary phase, allowing the flow of elute to continue. The binding efficiency of resins 250 depend not on the pH but of the functional group binding efficiency, where the selection of the function groups are important to what type of biological agents is the ideal product to remove from the blood stream. The key would be to aggregate in a local area and prevent large complex to use the circulatory system or lymphatic system from traveling in the blood and spread to another organ site.

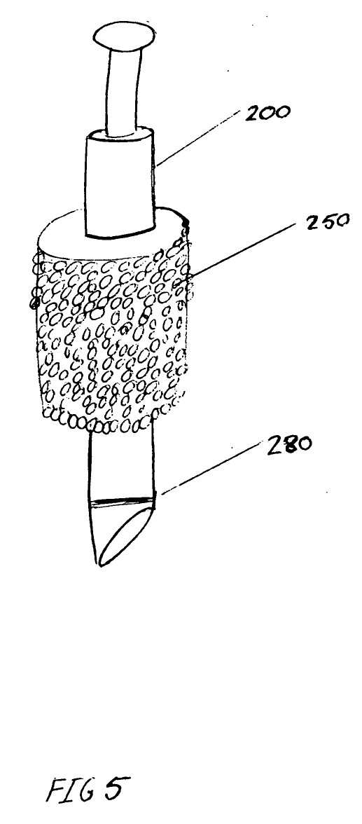

FIG. 5 illustrate the device with a telescoping microtubule 200 structure that is a collapsible antenna device where as the physical movement of the needle can be inserted into a stationary sleeve and only the needle internal parts are moveable in and out of the body, so that the actual penetration of the skin is less in diameter and the removed analytes or toxins migrate out of the skin. This allows local removal of biologicals that have complexes bound to the resins.

An alternative embodiment utilizes the theory of moving bound system in conjunction with isofocusing where as the amphilytes or immobilines (Amersham-products) are attached to the (silica) beads and become the stationary phase, where the charges particles in the body move in the direction based on its charge to a zwitterions “state of net charge zero” whereby this is designed to be outside the body by optimizing the flow by the creating specific gradient of the fixed amphilytes on the bead. The gradient changes according to the target product and the isoelectric focusing point, which is important to note that the amphylites are fixed onto the silica bead using bind a silane derivative.

These preferred embodiments are intended to illustrate the principles of the invention, but not to limit its scope. Variations and alternate embodiments will be apparent to those skilled in the art and may be made without departing from the spirit and scope of the invention defined by the following claims.

We claim:

1. A device for transporting agents and analytes across tissues comprising: a patch-like structure with a voltaic cell that creates a polar directional field between an insulating membrane adhered to the tissue of a host and at least one reservoir that stores agents or collects analytes for trans-dermal transfer to the tissue through electrophoresis migration.

2. The device of claim 1, wherein at least one reservoir is attached to at least one microtubule suitable for transferring and removing agents to and from the tissue transversing the insulating membrane for sub-dermal tissue implantation.

3. The device of claim 2, wherein at least one microtubule has an anode charged ring.

4. The device of claim 2 wherein at least one microtubule has internal resin beads.

5. The device of claim 2 wherein at least one microtubule has external resin beads.

6. The device of claim 2 wherein at least one microtubule is telescopic.

7. The device of claim 1, wherein the analyte specifically binds to cations in the reservoir.

8. The medical device of claim 1, further comprising an external voltage measuring device attached to the reservoir and a wireless transmitter for communicating patient data to external sources.

9. The medical device of claim 8, wherein the external sources are computer databases.

10. The medical device of claim 1, further comprising: a reservoir having a therapeutic agent therein; and a therapeutic agent migration based on charge, capable of controlling release of a therapeutic agent from a reservoir in response to an instruction from the biometric recognition device.

11. The medical device of claim 1 wherein the analytes can be blood or serum containing, or some serum, platelets, leukocytes, sickle cell, white blood cells or unwanted agents of toxins, metal, lead, uric crystal formations, severe allergy reaction of histamines or unwanted biolytic agents introduced to the body.

12. The medical device of claim 1 wherein the voltaic cell is a photo-voltaic cell.

13. The medical device of claim 1 wherein the analyte in the bodily fluid can bind or react with the biological agent or therapeutic agent.

14. The medical device of claim 1 wherein the analyte can be insulin and the agent is a bioactive agent that is an antibody specific for insulin.

15. The medical device of claim 1 wherein the analyte is glucose and the agent is a bioactive agent that is an antibody specific for glucose.

16. The medical device of claim 1 wherein the analyte is glucose and the therapeutic agent is insulin.

17. The medical device of claim 1 wherein the analyte is the same as the therapeutic agent.

18. The medical device of claim 1 wherein the analyte is a metabolite of the therapeutic agent

19. A solar powered device that transports agents in and out of tissue by electrophoresis migration.
